(19) United States
(12) Patent Application Publication
(10) Pub. No.: US 2026/0144802 A1
(43) Pub. Date: May 28, 2026

(54) POST-OPERATIVE DELIRIUM SUPPRESSANT

(71) Applicants: NATIONAL UNIVERSITY CORPORATION KOCHI UNIVERSITY, Kochi (JP); Shionogi & Co., Ltd., Osaka (JP)

(72) Inventors: Bun AOYAMA, Kochi (JP); Takashi KAWANO, Kochi (JP)

(73) Assignees: NATIONAL UNIVERSITY CORPORATION KOCHI UNIVERSITY, Kochi (JP); Shionogi & Co., Ltd., Osaka (JP)

(21) Appl. No.: 19/121,537

(22) PCT Filed: Oct. 20, 2023
(86) PCT No.: PCT/JP2023/037999

§ 371 Date: Apr. 16, 2025

(30) Foreign Application Priority Data
Oct. 21, 2022 (JP) 2022-168884

(51) Int. Cl. A61K 31/573; A61K 9/00; A61K 31/568; A61K 31/575; A61K 31/58; A61P 25/02

(52) U.S. Cl. CPC A61K 31/573; A61K 9/0053; A61K 31/568; A61K 31/575; A61K 31/58; A61P 25/02

(57) ABSTRACT

An object of the present invention is to provide a formulation capable of effectively suppressing post-operative delirium. A post-operative delirium suppressant according to the present invention contains a GABA-A receptor-selective positive allosteric modulator as an active ingredient.

### TECHNICAL FIELD

The present invention relates to a formulation capable of effectively suppressing post-operative delirium.

### BACKGROUND ART

Post-operative delirium is cognitive dysfunction, disturbance of attention, clouding of consciousness, perceptual disturbance, and the like caused by surgery or anesthesia. In post-operative delirium, a patient who has become calm once after surgery follows a unique course of acute and mixed onset of “hypoactive delirium” exhibiting a depressive state in addition to confusion, hallucinations, and delusions defined as “hyperactive delirium” after about several hours to three days after surgery, and gradual settling for about one week. There is also “mixed delirium” that repeats hyperactive delirium and hypoactive delirium, and prevention and treatment are very often difficult. For example, post-operative delirium occurs in a recovery period after surgery, and when hyperactive delirium occurs, it interferes with post-operative nursing and care, such as pulling out a tube that is important for life support or moving around loudly at night, and it also disturbs surrounding patients, and further, a risk of falling down or falling off of the patient himself increases.

In recent years, hypoactive delirium, with which the patient seems to be settled at a glance, is regarded as important, and since the patient exhibits a depressive state, an oral intake of essential food and drink decreases after surgery, recovery of a nutritional state does not proceed, rehabilitation is prolonged, the number of hospital stays is prolonged, and, in turn, the patient may be life-threatening, such as complication of aspiration pneumonia. In addition, it has been pointed out that once post-operative delirium occurs, it develops into post-operative dementia, and even if surgery is successful, a decline in cognitive function may significantly impair quality of life thereafter. Therefore, prevention and treatment of post-operative delirium are urgent problems to be solved in a super-aging society.

There are also data that post-operative delirium occurs in 10 to 20% of patients after minimally invasive surgery, in 35% of patients after cardiac and macrovascular surgery, and in 80% of patients requiring post-operative intensive care. In particular, the incidence is higher in elderly patients of 70 years old or older, patients with a history of delirium, patients with dementia, patients with alcoholism, and the like.

For prevention of post-operative delirium, non-drug therapy is mainly used, such as avoidance of use of drugs that are post-operative risk factors, such as antiemetics, antihistamines, narcotics, benzodiazepines, and antiparkinsonian drugs, in addition to optimization of general conditions such as respiration and circulation, sufficient analgesia, and maintenance of cognitive function. For hyperactive post-operative delirium, antipsychotic drugs such as haloperidol may be used, but it cannot be said that drug therapy has been sufficiently established. Furthermore, drug therapy for hypoactive post-operative delirium, which has been regarded as important in recent years as described above, has not yet been established.

As described above, post-operative delirium is often observed particularly in elderly patients, and recently, for example, in a case of post-operative obscene incidents by a mammary gland surgeon, the presence or absence of post-operative delirium is one of the focuses, and a technique for preventing or treating post-operative delirium is required. However, it is difficult to say that a method for preventing and treating post-operative delirium is sufficiently established, and even a candidate therapeutic agent does not exist for hypoactive post-operative delirium.

For example, Patent Document 1 describes use of a pregnenolone derivative for treatment of cognitive dysfunction, and exemplifies delirium as cognitive dysfunction. However, in Patent Document 1, there is no description of post-operative delirium, and of course, there is no experimental result regarding post-operative delirium. Patent Document 1 describes experimental data showing that the pregnenolone derivative does not modify binding of any of 85 receptors including a gamma-aminobutyric acid (GABA) receptor.

In addition, Patent Document 2 describes that a GABA-A receptor-selective positive allosteric modulator is used for treatment of symptoms in perimenopause or late menopause, and Patent Document 3 describes that a GABA-A receptor-selective positive allosteric modulator is used for treating diseases such as depression associated with epilepsy and other central nervous system disorders.

### PRIOR ART REFERENCES

### Patent Document

Patent Document 1 JP 2022-515373 A
Patent Document 2 JP 2022-524505 A
Patent Document 3 JP 2022-532710 A

### SUMMARY OF THE INVENTION

### Problems to be Solved by the Invention

As described above, in recent years, post-operative delirium has become a problem, but a method for preventing or treating post-operative delirium has not been sufficiently established yet.

Therefore, an object of the present invention is to provide a formulation capable of effectively suppressing post-operative delirium.

### Means for Solving the Problem

In order to solve the above problem, the present inventors have intensively studied, and in particular, constructed an aged post-operative delirium model rat in which attention and cognitive abilities, which are essential for diagnosis of post-operative delirium, can be evaluated, and searched for a drug effective for suppressing post-operative delirium using this rat. As a result, the present inventors have found that a GABA-A receptor-selective positive allosteric modulator is effective for suppressing post-operative delirium, thereby completing the present invention.

Hereinafter, the present invention will be described.

[1] A post-operative delirium suppressant, comprising a GABA-A receptor-selective positive allosteric modulator as an active ingredient.

[2] The post-operative delirium suppressant according to [1], wherein the GABA-A receptor-selective positive allosteric modulator is a neuroactive steroid.

[3] The post-operative delirium suppressant according to [2], wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

[4] The post-operative delirium suppressant according to [2], wherein the neuroactive steroid is brexanolone and/or zuranolone.

[5] The post-operative delirium suppressant according to any one of [1] to [4], which is intravenously administered.

[6] The post-operative delirium suppressant according to any one of [1] to [4], which is orally administered.

[7] Use of a GABA-A receptor-selective positive allosteric modulator for suppression of post-operative delirium.

[8] The use according to [7], wherein the GABA-A receptor-selective positive allosteric modulator is a neuroactive steroid.

[9] The use according to [8], wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

[10] The use according to [8], wherein the neuroactive steroid is brexanolone and/or zuranolone.

[11] The use according to any one of [7] to [10], wherein the GABA-A receptor-selective positive allosteric modulator is intravenously administered.

[12] The use according to any one of [7] to [10], wherein the GABA-A receptor-selective positive allosteric modulator is orally administered.

[13] A method for suppressing post-operative delirium, comprising:
administering a GABA-A receptor-selective positive allosteric modulator as an active ingredient to a patient.
   [14] The method according to [13], wherein the GABA-A receptor-selective positive allosteric modulator is a neuroactive steroid.

[15] The method according to [14], wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

[16] The method according to [14], wherein the neuroactive steroid is brexanolone and/or zuranolone.

[17] The method according to any one of [13] to [16], wherein the GABA-A receptor-selective positive allosteric modulator is intravenously administered.

[18] The method according to any one of [13] to [16], wherein the GABA-A receptor-selective positive allosteric modulator is orally administered.

[19] Use of a GABA-A receptor-selective positive allosteric modulator for manufacture of a medicament for suppression of post-operative delirium.

[20] The use according to [19], wherein the GABA-A receptor-selective positive allosteric modulator is a neuroactive steroid.

[21] The use according to [20], wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

[22] The use according to [20], wherein the neuroactive steroid is brexanolone and/or zuranolone.

[23] The use according to any one of [19] to [22], wherein the GABA-A receptor-selective positive allosteric modulator is intravenously administered.

[24] The use according to any one of [19] to [22], wherein the GABA-A receptor-selective positive allosteric modulator is orally administered.

[25] A GABA-A receptor-selective positive allosteric modulator for suppression of post-operative delirium.

[26] The GABA-A receptor-selective positive allosteric modulator according to [25], wherein the GABA-A receptor-selective positive allosteric modulator is a neuroactive steroid.

[27] The GABA-A receptor-selective positive allosteric modulator according to [26], wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

[28] The GABA-A receptor-selective positive allosteric modulator according to [26], wherein the neuroactive steroid is brexanolone and/or zuranolone.

[29] The GABA-A receptor-selective positive allosteric modulator according to any one of [25] to [28], wherein the GABA-A receptor-selective positive allosteric modulator is intravenously administered.

[30] The GABA-A receptor-selective positive allosteric modulator according to any one of [25] to [28], wherein the GABA-A receptor-selective positive allosteric modulator is orally administered.

### Effect of the Invention

According to the method of the present invention, post-operative delirium can be effectively prevented and treated. Therefore, the present invention is industrially very excellent as a technique capable of suppressing post-operative delirium, which has become a problem in recent years.

### BRIEF DESCRIPTION OF THE DRAWINGS

FIG. 1 is a result of comparison between surgical groups (solvent administration group and ALLO administration group) and non-surgical groups (control group and ALLO administration group). FIG. 1(1) is a graph showing a result of a behavior test regarding attention (proportion of freezing behavior time) of post-operative delirium model rats, and FIG. 1(2) is a graph showing a result of a behavior test regarding cognitive abilities.

FIG. 2 is a result of comparison between surgical groups (solvent administration group and ALLO administration group) and non-surgical groups (control group and ALLO administration group). FIG. 2(1) is a graph showing a relative expression level of IL-1β mRNA in the hippocampus of post-operative delirium model rats, and FIG. 2(2) is a graph showing a relative expression level of TNFα mRNA.

FIG. 3 is a result of comparison between surgical groups (solvent administration group and ALLO administration group) and non-surgical groups (control group and ALLO administration group). FIG. 3(1) is a graph showing an expression level of IL-1β in the hippocampus of post-operative delirium model rats, and FIG. 3(2) is a graph showing an expression level of TNFα.

FIG. 4 is a result of comparison between surgical groups (solvent administration group and ALLO administration group) and a non-surgical group (control group). FIG. 4(1) is a graph showing a relative expression level of IL-1β mRNA in the hippocampus of post-operative delirium model rats, and FIG. 4(2) is a graph showing a relative expression level of TNFα mRNA.

FIG. 5 is a result of comparison between a control group in which no surgery was performed, and a solvent administration group, a brexanolone administration group, and a zuranolone administration group in which surgery was performed, and a graph showing a relative expression level of IL-1β mRNA in the hippocampus of post-operative delirium model rats.

### MODE FOR CARRYING OUT THE INVENTION

A post-operative delirium suppressant according to the present invention contains a GABA-A receptor-selective positive allosteric modulator as an active ingredient. Hereinafter, the GABA-A receptor-selective positive allosteric modulator may be abbreviated as “GABA-A PAM”. In the present invention, “suppression” includes a concept of “treatment” for suppressing post-operative delirium after onset of post-operative delirium and a concept of “prevention” for suppressing the onset itself of post-operative delirium.

γ-Aminobutyric acid (GABA) is a major suppressive neurotransmitter in a mature central nervous system, and GABA receptors include an ionotropic GABA-A receptor and a metabolic GABA-B receptor. Each subunit of the GABA-A receptor is a quadruple transmembrane protein, and as in a case of a glycine receptor, each subunit forms a pentamer centered at a second transmembrane site (M2) and forms an anion channel in the central part. As the GABA-A receptor, 19 types of subunits are known. In general, drugs that enhance GABA-A receptor function, such as benzodiazepine drugs, are used as therapeutic agents for anxiety, insomnia, and the like. Thus, GABA-A PAM, a selective positive modulator of the GABA-A receptor, may stabilize the mind and suppress post-operative delirium.

GABA-A PAM positively modulates the GABA-A receptor by binding to an allosteric site that is not a GABA-binding site of the GABA-A receptor. Examples of GABA-A PAM include neuroactive steroids. The neuroactive steroid is an endogenous or exogenous steroid that rapidly changes excitability of a nerve by an interaction with a ligand-dependent ion channel or a receptor on a cell surface. In the present invention, the neuroactive steroid refers to an inhibitory neurosteroid that acts particularly as a positive allosteric modulator of GABA and exhibits actions such as an antidepressant action, an anxiolytic action, a stress reducing action, a reward action, a prosocial action, an antiaggressive action, a sexual desire promoting action, a sedative action, a sleep promoting action, a cognitive/memory eliminating action, an analgesic action, an anesthetic action, an anticonvulsant action, a neuroprotective action, and a neurogenic action.

Examples of the neuroactive steroid include one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof, and in particular, brexanolone and/or zuranolone is preferable. Brexanolone and zuranolone have the following chemical structures, respectively. Brexanolone is also referred to as allopregnanolone, which is also abbreviated as ALLO, and zuranolone is one of allopregnanolone derivatives.

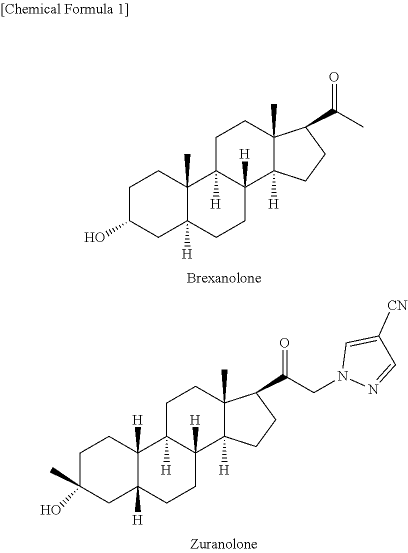

As the neuroactive steroid, in particular, zuranolone or a salt thereof is preferable. Since the neuroactive steroid is generally administered by injection, it is essential to secure a venous blood vessel for an infusion line, but in post-operative delirium, injection administration itself on the premise of securing a venous blood vessel can be an inducing factor for post-operative delirium to be treated. On the other hand, zuranolone can be orally administered, and furthermore, it may be administered once a day. Therefore, it can be said that this point is a great advantage for suppressing post-operative delirium.

The neuroactive steroid may be a salt. Examples of the salt include inorganic acid salts such as hydrochloride, hydrobromide, hydroiodide, sulfate, nitrate, perchlorate, and phosphate; organic acid salts such as oxalate, malonate, maleate, fumarate, lactate, malate, citrate, tartrate, benzoate, trifluoroacetate, acetate, methanesulfonate, p-toluenesulfonate, and trifluoromethanesulfonate; and amino acid salts such as glutamate and aspartate.

A dosage form of the post-operative delirium suppressant according to the present invention is not particularly limited, may be appropriately adjusted according to GABA-A PAM as an active ingredient, and is not particularly limited. Examples thereof include, as oral formulations, tablets, powders, fine granules, capsules, sugar-coated tablets, granules, solutions, and syrups, and also include injections. Examples of the tablet include film-coated tablets, orally disintegrating tablets (OD tablets), sugar-coated tablets, chewable tablets, and sublingual tablets. Since the neuroactive steroid is generally administered by injection, the dosage form of the post-operative delirium suppressant according to the present invention may be an injection. However, as described above, since injection administration itself can be an inducing factor for post-operative delirium, an oral formulation is preferable. In the post-operative delirium suppressant according to the present invention, a pharmaceutically acceptable additive may be used according to the dosage form. Examples of such additives include excipients, disintegrants, lubricants, binders, suspending agents, fillers, diluents, viscosity agents, antioxidants, colorants, flavoring agents, sweeteners, coating agents, glidants, antiaggregation agents, preservatives, solubilizing agents for active ingredients, stabilizers, pH adjusting agents, and isotonizing agents.

The post-operative delirium suppressant according to the present invention contains GABA-A PAM as an active ingredient. The active ingredient refers to a component exhibiting a suppressive action on post-operative delirium among components contained in the post-operative delirium suppressant according to the present invention. In other words, the post-operative delirium suppressant according to the present invention contains GABA-A PAM in an amount that exhibits a suppressive effect on post-operative delirium. Specifically, although not particularly limited, for example, when the post-operative delirium suppressant according to the present invention is an injection, a proportion of GABA-A PAM can be about 0.1 mg/mL or more and 20 mg/mL or less. When the post-operative delirium suppressant according to the present invention is an oral formulation, the proportion of GABA-A PAM can also be 0.05% by mass or more and 20% by mass or less.

A method for suppressing post-operative delirium according to the present invention includes a step of administering a GABA-A receptor-selective positive allosteric modulator as an active ingredient to a patient. An administration frequency and a dose of the post-operative delirium suppressant according to the present invention may be appropriately adjusted according to age, sex, condition, and the like of a subject for administration, and an amount capable of exhibiting an anti-aging effect is administered to the subject for administration. For example, a dose of GABA-A PAM per administration can be 1 mg/kg body weight or more and 10 mg/kg body weight or less. The number of administrations per day is also not particularly limited, and can be, for example, 0.5 times/day or more and 3 times/day or less.

The post-operative delirium suppressant according to the present invention can be administered not only to humans but also to animals other than humans. Examples of animals for administration include livestock such as horses, cows, pigs, sheep, goats, camels, and llamas; competitive animals such as racehorses; pets such as dogs and cats; experimental animals such as mice, rats, guinea pigs, and rabbits; poultry such as chickens, ducks, turkeys, and ostriches.

The present application claims the benefit of priority based on Japanese Patent Application No. 2022-168884 filed on Oct. 21, 2022. The entire contents of the specification of Japanese Patent Application No. 2022-168884 filed on Oct. 21, 2022 are incorporated herein by reference.

### EXAMPLES

Hereinafter, the present invention will be described more specifically with reference to Examples, but the present invention is not limited by the following Examples as a matter of course, and can be implemented with appropriate modifications within a range that can be adapted to the gist described above and below, and all of them are included in the technical scope of the present invention.

### Example 1: Evaluation of Post-Operative Delirious Behavior by Trace Memory Conditioning Test

Forty (40) SD male elderly rats of 19 to 24 months of age were randomly divided into a control group, an ALLO administration group, a laparotomy+solvent administration group, and a laparotomy+ALLO administration group by 10 rats.

Rats in the laparotomy+solvent administration group and the laparotomy+ALLO administration group were anesthetized with 1.5 to 2.0% isoflurane, and then underwent a median incision of 2 cm in the abdomen. The small intestine was pulled out by about 5 cm from the laparotomy site and extended with a finger for 3 minutes. Next, the abdomen was closed, and local infiltration anesthesia was applied to the wound with 0.2% ropivacaine for post-operative pain. To rats in the laparotomy+ALLO administration group, 10 mg/kg of brexanolone was intraperitoneally administered immediately after surgery. To rats in the laparotomy+solvent administration group, only the same amount of solvent was administered instead of brexanolone.

Rats in the control group and the ALLO administration group were not subjected to laparotomy, and only subjected to anesthesia with isoflurane and local infiltration anesthesia with ropivacaine in the same manner. To rats in the ALLO administration group, 10 mg/kg of brexanolone was intraperitoneally administered in the same manner as in the laparotomy+ALLO administration group after local infiltration anesthesia with ropivacaine. To rats in the control group, only the same amount of solvent (35% hydroxypropyl-β-cyclodextrin) was administered instead of brexanolone.

Twelve (12) hours after that, for each group of rats, a sound source of 75 dB was used as a conditional stimulus and an electrical stimulus of 0.8 mA was used as an unconditional stimulus to evaluate attention and cognitive abilities, which are main symptoms of delirium. First, as memory acquisition, a total of 8 cycles of sound stimulation for 10 seconds, a trace period for 30 seconds, and electrical stimulation for 0.7 seconds were performed to acquire memory of stimulation.

Twelve (12) hours after acquisition of memory of stimulation, a “trace memory test” was performed. Specifically, “only sound stimulation” was performed under different gauges, and freezing behavior at that time was measured. When freezing behavior did not occur, it was determined that the memory could not be acquired, that is, attention was lacking.

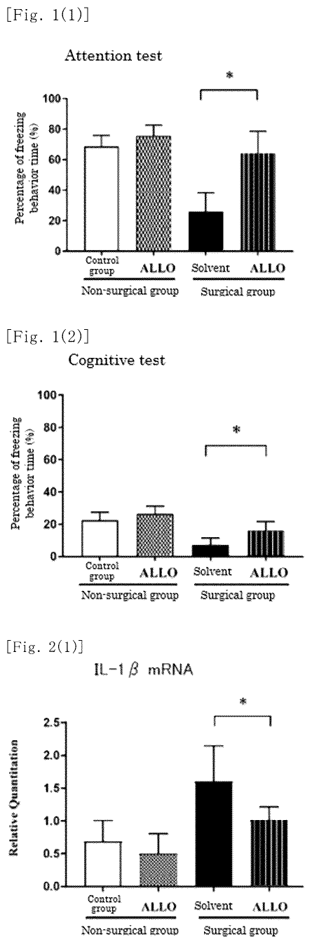

Finally, 4 hours after the trace memory test, rats were placed under a cage similar to the initial memory acquisition cage and freezing behavior of the rats without stimulation was measured as a “context memory test”. Here, when freezing behavior does not occur, it means that cognitive abilities are deteriorated. FIG. 1(1) shows a result of a behavior test regarding attention, and FIG. 1(2) shows a result of a behavior test regarding cognitive abilities. In FIG. 1, “*” indicates that there is a significant difference with p<0.05 in a Tukey's multiple comparison test after one-way analysis of variance.

As shown in FIG. 1, the attention and cognitive abilities of rats subjected to laparotomy and to which only the solvent was administered were clearly lower than those of rats that were not subjected to laparotomy.

On the other hand, it was proved that the attention and cognitive abilities of rats subjected to laparotomy and to which brexanolone was administered were significantly higher than those of rats subjected to laparotomy and to which only the solvent was administered.

As described above, it became clear that even after undergoing surgery, reduction in attention and cognitive abilities, which are main symptoms of post-operative delirium, can be improved, that is, post-operative delirium can be effectively suppressed by administration of allopregnanolone derivatives.

### Example 2: Expression Test of Inflammatory Cytokines Leading as Onset Mechanism of Post-Operative Delirium

Forty (40) SD male elderly rats of 19 to 24 months of age were randomly divided into a control group, an ALLO administration group, a laparotomy+solvent administration group, and a laparotomy+ALLO administration group by 10 rats.

Rats in the laparotomy group were subjected to laparotomy in the same manner as in Example 1. To rats in the laparotomy+ALLO administration group, 10 mg/kg of brexanolone was intraperitoneally administered immediately after surgery. To rats in the laparotomy+solvent administration group, only the same amount of solvent was administered instead of brexanolone.

Rats in the control group and the ALLO administration group were not subjected to laparotomy, and only subjected to anesthesia with isoflurane and local infiltration anesthesia with ropivacaine in the same manner. To rats in the ALLO administration group, 10 mg/kg of brexanolone was intraperitoneally administered in the same manner as in the laparotomy+ALLO administration group after local infiltration anesthesia with ropivacaine. To rats in the control group, only the same amount of solvent (35% hydroxypropyl-β-cyclodextrin) was administered instead of brexanolone.

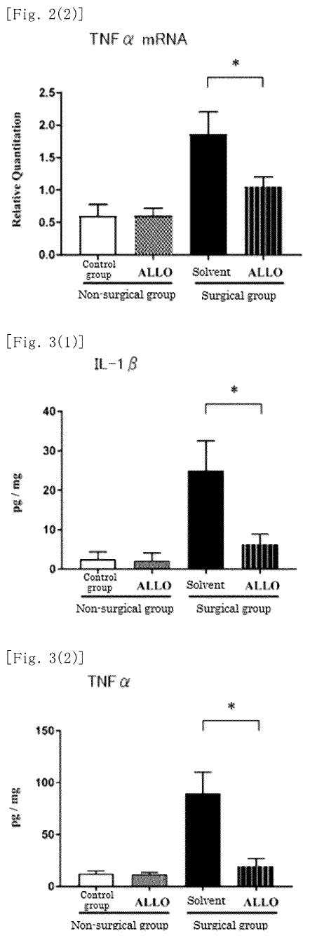

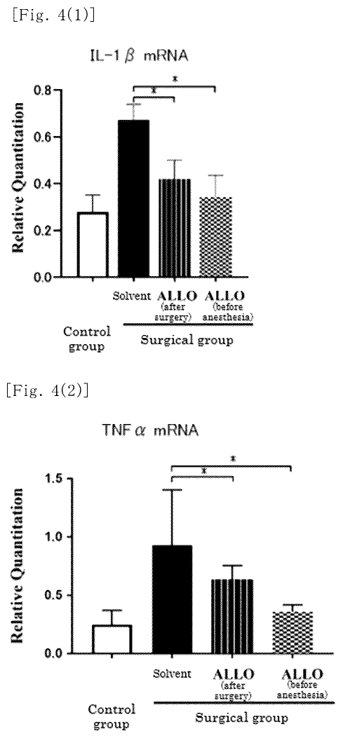

Twelve (12) hours after surgery, the hippocampus, which is considered to be strongly related to an onset of post-operative delirium, was extracted, and mRNA expression levels of IL-1β and TNFα, which are inflammatory cytokines suspected to be related to an onset mechanism of post-operative delirium, were measured by an RT-PCR method. After the trace memory conditioning test of Example 1, the hippocampus was extracted at 48 hours after the laparotomy, and expression levels of IL-1 and TNFα in the hippocampus sample were measured by an ELISA method. FIG. 2(1) shows a relative expression level of IL-1β mRNA, FIG. 2(2) shows a relative expression level of TNFα mRNA, FIG. 3(1) shows an expression level of IL-1β, and FIG. 3(2) shows an expression level of TNFα. In FIGS. 2 and 3, “*” indicates that there is a significant difference with p<0.05 with respect to the laparotomy+solvent administration group in a Tukey's multiple comparison test after one-way analysis of variance.

As shown in FIGS. 2 and 3, it was experimentally suggested that allopregnanolone derivatives can significantly reduce the expression of IL-1 and TNFα, which are inflammatory cytokines, in the brain after surgery, and suppress an onset of post-operative delirium.

### Example 3: Confirmation of Preventive Effect

Forty (40) SD male elderly rats of 19 to 24 months of age were randomly divided into a control group, a laparotomy+solvent pre-operative administration group, a laparotomy+ALLO pre-operative administration group, and a laparotomy+ALLO post-operative administration group by 10 rats.

Rats in the laparotomy group were subjected to laparotomy in the same manner as in Example 1. To rats in the laparotomy+ALLO pre-operative administration group, 10 mg/kg of brexanolone was intraperitoneally administered 30 minutes before anesthesia with isoflurane. To rats in the laparotomy+ALLO post-operative administration group, 10 mg/kg of brexanolone was intraperitoneally administered immediately after surgery. To rats in the laparotomy+solvent pre-operative administration group, only the same amount of solvent (35% hydroxypropyl-β-cyclodextrin) was administered instead of brexanolone 30 minutes before anesthesia with isoflurane.

To rats in the control group, laparotomy was not performed, and only the same amount of solvent (35% hydroxypropyl-β-cyclodextrin) was administered instead of brexanolone 30 minutes before anesthesia with isoflurane and local infiltration anesthesia with ropivacaine.

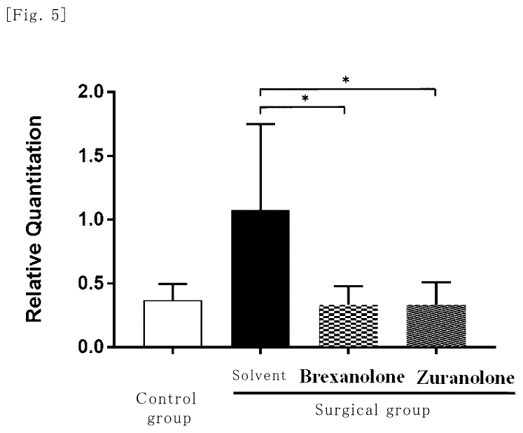

Twelve (12) hours after surgery, the hippocampus, which is considered to be strongly related to an onset of post-operative delirium, was extracted, and mRNA expression levels of IL-1β and TNFα, which are inflammatory cytokines suspected to be related to an onset mechanism of post-operative delirium, were measured by an RT-PCR method. FIG. 4(1) shows a relative expression level of IL-1β mRNA, and FIG. 4(2) shows a relative expression level of TNFα mRNA. In FIG. 4, “*” indicates that there is a significant difference with p<0.05 with respect to the laparotomy+solvent administration group in a Tukey's multiple comparison test after one-way analysis of variance.

As shown in FIG. 4, it was experimentally suggested that allopregnanolone derivatives can significantly reduce the expression of IL-1 and TNFα, which are inflammatory cytokines, in the brain after surgery, and suppress an onset of post-operative delirium even when the allopregnanolone derivatives are administered at any timing before and after surgery.

### Example 4: Comparison of Allopregnanolone Derivatives

Forty (40) SD male elderly rats of 19 to 24 months of age were randomly divided into a control group, a laparotomy+solvent pre-operative administration group, a laparotomy+brexanolone pre-operative administration group, and a laparotomy+zuranolone pre-operative administration group by 10 rats.

Rats in the laparotomy group were subjected to laparotomy in the same manner as in Example 1. To rats in the laparotomy+brexanolone pre-operative administration group, 10 mg/kg of brexanolone was intraperitoneally administered 30 minutes before anesthesia with isoflurane. To rats in the laparotomy+zuranolone pre-operative administration group, 3 mg/kg of zuranolone was orally administered 30 minutes before anesthesia with isoflurane. To rats in the laparotomy+solvent administration group, only the same amount of solvent (35% hydroxypropyl-β-cyclodextrin) was administered instead of allopregnanolone derivatives after surgery.

To rats in the control group, laparotomy was not performed, and only the same amount of solvent (35% hydroxypropyl-β-cyclodextrin) was administered instead of allopregnanolone derivatives 30 minutes before anesthesia with isoflurane and local infiltration anesthesia with ropivacaine.

Twelve (12) hours after surgery, the hippocampus, which is considered to be strongly related to an onset of post-operative delirium, was extracted, and an mRNA expression level of IL-1β, which is an inflammatory cytokine suspected to be related to an onset mechanism of post-operative delirium, was measured by an RT-PCR method. FIG. 5 shows a relative expression level of IL-1β mRNA. In FIG. 5, “*” indicates that there is a significant difference with p<0.05 with respect to the laparotomy+solvent administration group in a Tukey's multiple comparison test after one-way analysis of variance.

As shown in FIG. 5, it was experimentally suggested that allopregnanolone derivatives brexanolone and zuranolone both can significantly reduce the expression of IL-1β, which is an inflammatory cytokine, in the brain after surgery, and suppress an onset of post-operative delirium.

1-12. (canceled)

13. A method for suppressing post-operative delirium, comprising:
administering a GABA-A receptor-selective positive allosteric modulator as an active ingredient to a patient.

14. The method according to claim 13, wherein the GABA-A receptor-selective positive allosteric modulator is a neuroactive steroid.

15. The method according to claim 14, wherein the neuroactive steroid is one or more neuroactive steroids selected from brexanolone, zuranolone, tetrahydrodeoxycorticosterone, androstane, androstane 3α-androstanediol, cholestane cholesterol, pregnane, eltanolone, ganaxolone, and salts thereof.

16. The method according to claim 14, wherein the neuroactive steroid is brexanolone and/or zuranolone.

17. The method according to claim 13, wherein the GABA-A receptor-selective positive allosteric modulator is intravenously administered.

18. The method according to claim 13, wherein the GABA-A receptor-selective positive allosteric modulator is orally administered.
